(19) United States
(12) Patent Application Publication
(10) Pub. No.: US 2026/0162776 A1
(43) Pub. Date: Jun. 11, 2026

(54) Testing Routes Using Adaptive Search Retrieval to Obtain Convergence Outputs for Optimizing Ab Initio Solvers

(71) Applicant: QpiAI India Private Limited, Bangalore, Nagavara (IN)

(72) Inventors: Aswanth Krishnan, Bengaluru (IN); Lakshya Priyadarshi, Lucknow (IN); Nagendra Nagaraja, Bangalore (IN); Manjunath Ramachandrappa Venkatesh, Bangalore (IN)

(73) Assignee: QpiAI India Private Limited, Bangalore, Nagavara (IN)

(21) Appl. No.: 19/316,906

(22) Filed: Sep. 2, 2025

(30) Foreign Application Priority Data
Dec. 10, 2024 (IN) 202441097294

(51) Int. Cl. G16C 20/10; G16C 20/40; G16C 20/70; G16C 20/90

(52) U.S. Cl. CPC G16C 20/10; G16C 20/40; G16C 20/70; G16C 20/90

(57) ABSTRACT

In some embodiments, a device and related method may test reaction routes and generate synthesis processes. The method may include converting a query containing chemical reaction information into a vector for similarity searching in a vector database, obtaining solver parameters associated with similar past queries based on the vector, and testing proposed routes using a quantum chemistry solver to generate reaction characteristics. The method may also include selecting viable routes for use in generating one or more downstream process operations.

### CROSS-REFERENCE TO RELATED APPLICATIONS

This application claims the benefit of priority to Indian Provisional Application No. 202441097294, filed Dec. 10, 2024, the entirety of which is incorporated by reference herein.

### BACKGROUND

Ab initio solvers and related solvers play a crucial role in computational chemistry by calculating molecular properties and reaction pathways from first principles using quantum mechanics. These methods, along with other computational solvers like density functional theory (DFT) and semi-empirical approaches, enable the prediction of reaction mechanisms, transition states, and thermodynamic properties before conducting expensive and time-consuming synthesis operations. By providing detailed insights into electronic structure and molecular behavior, these computational tools accelerate chemical discovery, optimize reaction conditions, and help design new catalysts and materials with desired properties.

### SUMMARY

Computational solvers remain useful tools in modern chemistry, allowing the exploration of reaction landscapes, predict molecular properties, and guide experimental design with unprecedented detail and efficiency. However, these methods face significant obstacles that can limit their practical application, particularly issues related to computational accuracy where the choice of basis set, exchange-correlation functional, or level of theory can dramatically affect the reliability of results. Convergence problems present another major challenge, as iterative algorithms may fail to reach stable solutions for complex systems with challenging electronic structures, multiple conformations, or strong electron correlation effects. Additionally, the computational cost of high-accuracy methods often forces computer systems to balance precision and feasibility, especially when simulating operations involving large molecular systems or exploring extensive reaction networks.

Some embodiments may resolve such issues and other issues by performing a dynamic adaptive search to retrieve appropriate ab initio solvers useful for efficiently simulating chemical reaction routes. Some embodiments may generate a vector from a reaction query that contains information describing a set of chemical reaction routes, with this information including details on reactants and reaction conditions. Some embodiments may then perform a similarity search within a test query vector database using the generated vector to identify stored records and retrieve quantum chemistry solver parameters associated with previous reaction test queries that meet threshold energy gap criteria. Furthermore, some embodiments may execute a quantum chemistry solver using the retrieved quantum chemistry solver parameters to evaluate the chemical reaction routes and produce a set of outputs that include reaction rate constants for an end product and a byproduct. Additionally, some embodiments may store a pruned set of reaction routes in a reaction route database after selecting viable reaction pathway models by pruning the initial set of chemical reaction routes based on the generated reaction rate constants. Finally, some embodiments may update the vector database by incorporating a representation of the pruned reaction route set if a first energy gap found in the outputs is determined to satisfy a convergence-based threshold indicating energy gap convergence.

By performing operations and using related systems described in this disclosure, some embodiments may enhance testing accuracy and reproducibility for simulations involving chemical reaction routes and processes developed from these routes. Some embodiments may employ ab initio solver systems in conjunction with adaptive search retrieval techniques to efficiently identify and configure optimal parameter sets for reaction modeling. Furthermore, some embodiments may increase the efficiency of analyzing chemical reaction routes across varied testing scenarios in computational chemistry.

Various other aspects, features, and advantages of the invention will be apparent through the detailed description of the invention and the drawings attached hereto. It is also to be understood that both the foregoing general description and the following detailed description are examples and are not restrictive of the scope of the invention.

### BRIEF DESCRIPTION OF THE DRAWINGS

FIG. 1 shows an example system for testing reaction routes and generating synthesis processes based on the reaction routes, in accordance with one or more embodiments.

FIG. 2 shows an example conceptual diagram of a system that includes different infrastructure or application layers used to perform adaptive search retrieval when using ab initio solvers, in accordance with one or more embodiments.

FIG. 3 depicts a conceptual architecture of a system for testing reaction routes using adaptive search retrieval to obtain synthesis process results, in accordance with one or more embodiments.

FIG. 4 shows a flowchart of a process for testing reaction routes and generating synthesis processes, in accordance with one or more embodiments.

The technologies described herein will become more apparent to those skilled in the art by studying the detailed description in conjunction with the drawings. Embodiments of implementations describing aspects of the invention are illustrated by way of example, and the same references can indicate similar elements. While the drawings depict various implementations for the purpose of illustration, those skilled in the art will recognize that alternative implementations can be employed without departing from the principles of the present technologies. Accordingly, while specific implementations are shown in the drawings, the technology is amenable to various modifications.

### DETAILED DESCRIPTION OF THE DRAWINGS

In the following description, for the purposes of explanation, numerous specific details are set forth in order to provide a thorough understanding of the embodiments of the invention. It will be appreciated, however, by those having skill in the art that the embodiments of the invention may be practiced without these specific details or with an equivalent arrangement. In other cases, well-known structures and devices are shown in block diagram form in order to avoid unnecessarily obscuring the embodiments of the invention.

While numerous examples provided herein describe embodiments in the context of chemical synthesis, computational chemistry, molecular modeling, or related domains, this is for purposes of illustration only. The systems, methods, architectures, and operations described are not limited to chemistry-specific data, solvers, or workflows. Unless expressly stated otherwise, references to “chemical reaction routes,” “reactants,” “quantum chemistry solvers,” or other chemistry-related terms should be construed broadly to encompass any analogous entities, processing steps, predictive models, or domain-specific solvers in other technical fields. For example, the same vector-based similarity search, solver parameter retrieval, pruning, caching, and workflow orchestration methods may be applied to domains including, but not limited to, mechanical design, materials science, genomics, process optimization, manufacturing workflows, logistics planning, or any domain in which data or process steps can be represented as structured or unstructured inputs and processed via computational models. Such generalizations are within the scope of the appended claims.

Even after identifying the desired molecular target through computational design, natural product isolation, or other discovery methods, the actual synthesis of complex organic molecules presents formidable challenges that can require years of intensive research and development. The complexity stems from the need to construct intricate molecular frameworks with precise control over stereochemistry, regioselectivity, and functional group compatibility, while developing synthetic routes that are both efficient and practical for the intended scale of production. Many target molecules contain challenging structural features such as strained ring systems, multiple chiral centers, densely functionalized cores, or chemically sensitive moieties that demand creative synthetic strategies and careful optimization of reaction conditions. Pharmaceutical compounds exemplify these challenges, as drug molecules often require not only successful laboratory synthesis but also scalable manufacturing processes. These factors can add years to the development timeline even after the synthetic route has been established.

Some embodiments may help address synthesis challenges by converting reactant and chemical reaction route data into vector representations and performing similarity searches to retrieve optimal solver parameters from a vector database. Quantum chemistry solvers may then test candidate routes, generate reaction rate constants, and enable automatic pruning of viable reaction pathway models based on quantitative outputs. This adaptive approach may streamline selection and optimization of complex synthetic routes, improve efficiency, and support rapid development and scale-up for challenging molecules such as pharmaceutical compounds.

For example, some embodiments may receive a query containing data on drug precursor chemicals and a set of proposed manufacturing routes. Some embodiments may convert this information into a vector representation reflecting key process details and relationships. After generating the vector, some embodiments may search a vector database for relevant solver parameters, use quantum chemistry solvers to test manufacturing routes and generate rate constants, prune unviable processes, and update both reaction route and vector databases based on data associated with computed energy gaps to optimize later use of the solver in similar situations.

Some embodiments may convert a query containing data about a chosen set of reactants and a group of chemical reaction routes into a vector that captures structural and contextual features of the synthesis. Some embodiments may then use this vector to perform a similarity search in a vector database, identifying stored records and retrieving quantum chemistry solver parameters from previous reaction test queries that produced energy gaps within specified thresholds. A computer system may, for instance, process a drug synthesis query featuring the production of an active pharmaceutical ingredient from known precursors, and extract solver parameter sets used in similar previous manufacturing scenarios with successful energetic profiles. Subsequent operations may involve testing the chemical reaction routes using the quantum chemistry solver with those parameters, generating reaction rate constants, storing pruned viable routes in the reaction route database, and updating vector representations based on outputs meeting a convergence-based standard.

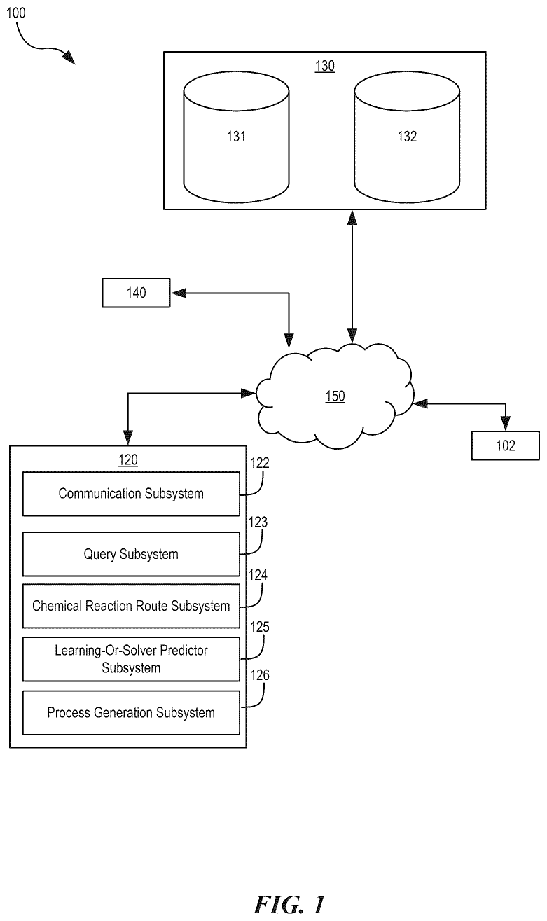

FIG. 1 shows an example system 100 for determining reaction routes and generating synthesis processes based on the reaction routes, in accordance with one or more embodiments. A system 100 includes a client device 102 in communication with a server 120 via a network 150. The system 100 includes various types of electronic devices, such as the client device 102. The client device 102 may include one of various types of computer devices usable as a client-side device, such as a laptop, data terminal, mobile computing device, etc. The client device 102 may send requests, responses, or other messages to the server 120 that may require communication with other computing devices or other electronic devices. Additionally, the server 120 may include various types of computing units, such as physically separate servers, virtual nodes hosted on one or more physical machines, or nodes on a cloud computing system. Applications, services, or other operations may use data provided by the client device 102, the server 120, a distributed cache system 140, or a set of databases 130 that includes a first networked database 131 and a second networked database 132. The set of databases 130 may include various types of databases, such as SQL databases, no SQL databases, graph databases, etc. In some embodiments, the server 120 may perform one or more operations related to a communication subsystem 122, a query subsystem 123, a chemical reaction route subsystem 124, a learning-or-solver subsystem 125, or a process generation subsystem 126.

In some embodiments, the communication subsystem 122 may obtain program instructions, commands, parameters, values, or other data from the server 120 or the set of databases 130. For example, the communication subsystem 122 may retrieve parameters such as solver parameters, learning model parameters, or additional query parameters from the set of databases 130. Furthermore, operations performed by the server 120 may use the communication subsystem 122 to send messages to the set of databases 130, the server 120, or another computing device described in this disclosure. For example, the server 120 uses the communication subsystem 122 to submit vector-based queries to the set of databases 130 that cause the set of databases 130 to retrieve queries. Furthermore, some embodiments may use the communication subsystem 122 to communicate with one or more remote computing devices to offload some or all of the operations described in this disclosure.

In some embodiments, the query subsystem 123 may obtain a query that includes data indicating a reactant or a chemical reaction route with data sourced from a user interface of a client device 102. For example, the query subsystem 123 may receive, via a user-generated query or a machine-generated query, specific elements such as molecular identifiers, reaction pathway descriptors, or structural formulas. Furthermore, some embodiments may construct queries based on the outputs of one or more components and previously provided queries. For example, a computer system may construct a query using possible output reactions from a chemical reaction route predictor and queries that were previously provided to the reaction route predictor by the client device 102. The query subsystem 123 may receive computational results indicating reaction feasibility and combine those results with historical query data submitted by the client device 102 to generate a query vector for downstream use (e.g., for use to select parameters for a learning model or solver).

Furthermore, some embodiments may use a reaction database and a computation engine to the query subsystem 123 to facilitate communication and action between the query subsystem 123, and a database, such a reaction database. For instance, the query subsystem 123 may parse and route a received chemical reaction query to form an input for a learning model or solver. Additionally, some embodiments may use the query subsystem 123 to interact with remote laboratory information management systems or external chemical databases to offload, support, or extend query interpretation and data retrieval operations described herein.

In some embodiments, the query subsystem 123 may turn a query into a query vector by encoding the information present in a user-generated or system-generated query into a structured numerical representation suitable for database search or machine learning operations. This transformation may be achieved by mapping query features such as chemical identifiers, structural data, or procedural text into high-dimensional embeddings using predefined algorithms, natural language models, or specialized cheminformatics techniques. For instance, some embodiments may use a molecular fingerprinting algorithm to represent reactant and product structures as numerical vectors. Alternatively, some embodiments may use neural network encoders (e.g., neural network encoders trained on chemical reaction corpora) to generate semantically rich query vectors that reflect contextual information.

As an example, the query subsystem 123 may process a query indicating a target molecule and an initial set of reactants. The query subsystem 123 may extract chemical structure descriptors from the query, encode the target molecule and each reactant using a deep learning-based chemical encoder, and then concatenate these embeddings to form a composite query vector. This query vector may represent the overall transformation or synthetic goal and may be suitable for database matching, parameter retrieval, or reaction outcome prediction. For example, the query subsystem 123 may use the query vector to obtain parameters for learning model operations or solver operations.

Some embodiments may perform adaptive search retrieval to perform more targeted searches to obtain appropriate model parameters, solver parameters, or other parameters for downstream operations. For example, some embodiments may generate a chain of vectors based on previous queries, previous query results, or previous vectors, where downstream vectors may be more accurately retrieve relevant parameters. As a more specific example, the query subsystem 123 may receive a query indicating a target molecule and an initial set of reactants and may encode the target and reactants into a query vector and provide this query vector to a retrosynthetic planner. The retrosynthetic planner may use the vector to determine an initial set of reactions necessary to produce the target molecule. Following this, the query subsystem 123 may generate a second vector by encoding the resulting set of reactions, thereby capturing both the proposed reaction pathways and the underlying chemical context. Some embodiments may then use this second vector for downstream analysis, such as using the vector as input for a learning model or to retrieve configuration parameters for an ab initio quantum chemistry solver to filter the initial set of chemical reaction routes into a pruned set of reaction routes.

In some embodiments, the chemical reaction route subsystem 124 may perform operations to generate information indicating a set of chemical reaction routes. In some embodiments, the information may include detailed data regarding reactants and associated reaction conditions. For example, the chemical reaction route subsystem 124 may analyze inputs describing a target compound and may retrieve a collection of viable synthetic pathways from a reaction database. Each reaction pathway may include a list of required reactants and specific conditions such as temperature, pressure, solvent selection, or catalyst presence. As another example, the chemical reaction route subsystem 124 may use machine learning outputs to propose alternative reaction routes. The proposed routes may detail each reactant necessary for every step and may include recommended concentrations and reaction times, where such parameters may be used for constructing downstream chemical production processes.

In some embodiments, the learning-or-solver subsystem 125 may use a learning model to predict chemical reaction routes when a query vector is sufficiently close to one or more known query vectors associated with successful learning model predictions. For example, if the query vector [0.42, 0.85, 0.13, 0.54, 0.77, 0.90] is be found within a predefined threshold distance from stored vectors such as [0.41, 0.83, 0.15, 0.56, 0.78, 0.89] or [0.44, 0.82, 0.10, 0.53, 0.76, 0.91], where each of these stored vectors are each linked to past queries and corresponding outputs, the learning-or-solver subsystem 125 may predict a pruned set of chemical reaction routes using a learning model.

In some embodiments, the learning model to be used for chemical reaction route prediction may have been trained using the past queries. Alternatively, or additionally, the learning model to be used for chemical reaction route prediction may receive, as part of the input, past query or corresponding past result. Furthermore, different learning models or different parameters with which to configure a learning model may be selected by the learning-or-solver subsystem 125 based on which stored vector(s) are nearest to the query vector, increasing the likelihood of obtaining contextually accurate predictions informed by historical outcomes.

In some embodiments, a learning model may take either a query vector or data related to the query vector, such as a chemical structure representing a reactant or reaction product, as an input to produce predicted chemical reaction routes. A learning model may include various types of models, such as graph neural networks, transformer-based models, sequence-to-sequence neural architectures, or ensemble machine learning methods trained specifically on reaction pathway data. For example, after the learning-or-solver subsystem 125 receives a query vector such as [0.39, 0.80, 0.17, 0.51, 0.74, 0.93] derived from an encoded reactant set and target structure, a graph neural network may output, as a representation of a chemical reaction route, a set of predicted chemical transformation steps leading to the desired product and environmental conditions appropriate for these transformation steps. As another example, a transformer model may take as input a chemical structure expressed as a SMILES string and, via attention-driven analysis, output the most plausible chemical reaction route, enabling rapid and reliable synthesis planning in computational chemistry applications.

In some embodiments, the learning-or-solver subsystem 125 may determine that there are no sufficiently close previous queries to a current query (e.g., the query vector does not have sufficiently close vectors in a vector database). In response, the learning-or-solver subsystem 125 may implement quantum chemistry solver parameters for ab initio solvers that are associated with one or more previous reaction test queries. This process may begin by accessing a parameter library stored in the set of databases 130, where historical solver parameters and reaction outcomes may be cataloged. A computer system may employ the learning-or-solver subsystem 125 to obtain these parameters by performing a similarity search in a vector database of the set of databases 130. The similarity search may use a query vector generated from the current reaction test query to identify a set of stored records with high semantic relevance. Some embodiments may use the query vector to find a set of relevant records that are not sufficiently close enough (e.g., within a maximum distance threshold in the vector space) to permit the use of a learning model, but sufficiently close enough to permit use of solver parameters associated with those learning models.

After identifying a stored set of relevant records using a query or corresponding query vector (or other data related to the query), the learning-or-solver subsystem 125 may retrieve corresponding quantum chemistry solver parameters for subsequent implementation. For example, some embodiments may retrieve parameters for physics-based quantum solvers, such as those based on density functional theory or Hartree-Fock methods, as well as parameters for other types of solvers utilizing machine learning models or heuristic algorithms. Such parameters may include basis set selections, convergence thresholds, initial electron configurations, computational grid settings, and specific algorithmic flags tailored for either ab initio calculations or data-driven estimation techniques. Additionally, the learning-or-solver subsystem 125 may select parameters for hybrid solvers that integrate traditional quantum mechanical models with predictive approaches. These retrieved parameters may enable the learning-or-solver subsystem 125 to perform accurate simulations and analyses across a broad spectrum of chemical phenomena. Such operations may help overcome the onerous computational cost of using sub-optimal solver parameters in ab initio solvers or other solvers.

In some embodiments, the learning-or-solver subsystem 125 may generate outputs that include energy gaps or may be used to determine energy gaps. For example, the learning-or-solver subsystem 125 may generate outputs that include calculated energy values for the reactants and products of a chemical reaction. These outputs may be used to determine energy gaps by computing the difference between the total electronic energy of the reactants and the products. Then learning-or-solver subsystem 125 may then selectively store the configuration parameters used by the solver based on a determination that the energy gap satisfies an energy gap threshold after solver convergence is reached. By storing the configuration parameters or indicating their utility, some embodiments may increase the efficiency of later use of a solver.

As an example, the learning-or-solver subsystem 125 may use retrieved quantum chemistry solver parameters to configure a solver that then outputs the reactant energies as −312.58 kJ/mol and the product energies as −435.21 kJ/mol. The learning-or-solver subsystem 125 may then determine the energy gap to be −122.63 kJ/mol, which may indicate the thermodynamic feasibility or stability of the reaction pathway. Furthermore, the learning-or-solver subsystem 125 may determine that the energy gap of −122.63 kJ/mol satisfies an energy gap threshold of −50 kJ/mol, indicating that the corresponding reaction configuration demonstrates sufficient thermodynamic favorability to warrant storing the configuration parameters in the set of databases 130 for future retrieval and analysis. It should be understood that satisfying an energy gap threshold means that an energy gap caused by a reaction is less than an energy gap threshold (e.g., −122.63 kJ/mol satisfies the energy gap threshold of −50 kJ/mol because −122.63 kJ/mol is less than −50 kJ/mol).

In some embodiments, the learning model may provide a confidence score associated with one or more other outputs (e.g., a predicted set of chemical transformation steps). The learning-or-solver subsystem 125 may compare this confidence score with a confidence threshold. In response to a result of this comparison indicating that the confidence score does not satisfy the confidence threshold (i.e., the confidence is too low), the learning-or-solver subsystem 125 may perform the operations described above to retrieve quantum chemistry solver parameters and execute a quantum chemistry solver using the retrieved parameters. Furthermore, in some embodiments, a user may change the confidence threshold. Alternatively, or additionally, a machine learning model may modify the confidence threshold based on detecting that a particular confidence value is associated with a sufficiently accurate result.

Some embodiments may use the process generation subsystem 126 to generate one or more synthesis process operations based on a set of reaction routes (e.g., a pruned set of reaction routes generated by the learning-or-solver subsystem 125). The process generation subsystem 126 may construct a directed workflow specifying operations such as individual reaction steps, purification protocols, and condition optimizations tailored to the selected route. For example, the process generation subsystem 126 may receive a pruned reaction route involving three steps for synthesizing a target compound and specify operations that include reagent addition, temperature control, and product isolation for each step. As another example, the subsystem may integrate machine learning predictions by filtering proposed operations to favor those with high predicted yields and safety profiles. Furthermore, the process generation subsystem 126 may query external databases or expert systems to confirm the availability of key precursors before finalizing the synthesis process workflow.

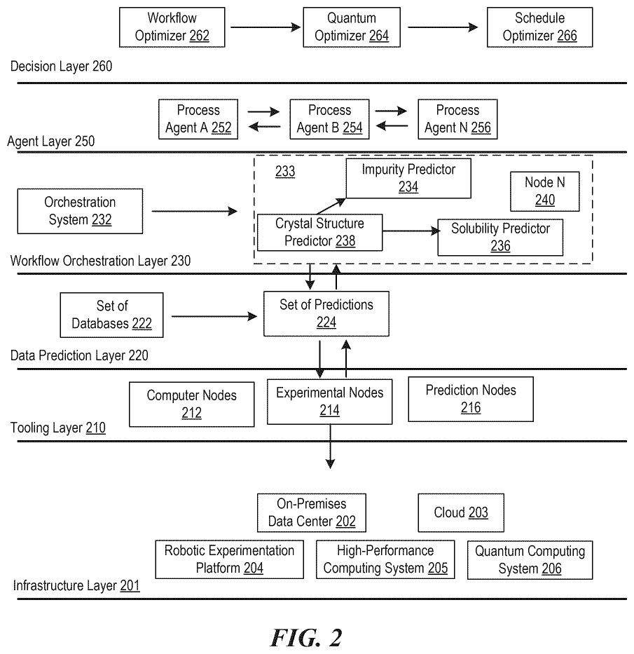

FIG. 2 shows an example conceptual diagram of a system 200 that includes different infrastructure or application layers used to perform adaptive search retrieval when using ab initio solvers, in accordance with one or more embodiments. The system 200 includes an infrastructure layer 201, which includes an on-premises data center 202, a cloud 203, a robotic experimentation platform 204, a high-performance computing system 205, and a quantum computing system 206. In some embodiments, the on-premises data center 202 may store proprietary reaction data, manage secure user credentials, and locally execute routine computational chemistry tasks or data retrieval operations within a controlled environment. In some embodiments, the cloud 203 may facilitate scalable data processing, enable remote access to shared datasets or machine learning models, and support the orchestration of distributed computational workflows across geographically separated users and resources. In some embodiments, the robotic experimentation platform 204 may automate laboratory synthesis, reaction monitoring, and sample processing by executing predefined or generated protocols. In some embodiments, the high-performance computing system 205 may run large-scale molecular simulations using one or more ab initio solvers, perform complex optimization tasks, or perform parallel retrosynthetic route evaluations. In some embodiments, the quantum computing system 206 may accelerate quantum chemistry calculations, solve optimization problems related to reaction pathways, or evaluate electronic structures and energy profiles with precision beyond classical computational limits.

The system 200 includes a tooling layer 210, which includes computer nodes 212, experimental nodes 214, and prediction nodes 216. In some embodiments, the computer nodes 212 may perform digital simulations, molecular modeling, or data analysis tasks such as retrosynthetic prediction, reaction feasibility assessment, and database querying. In some embodiments, the experimental nodes 214 may propose or execute laboratory experiments based on synthesized protocols, automate data collection from chemical instrumentation, and oversee activities such as reaction setup, product isolation, and analytical characterization. In some embodiments, the prediction nodes 216 may forecast reaction outcomes, estimate process yields, or quantify uncertainty in proposed synthesis routes using machine learning models and statistical inference validated against historical and experimental data, supporting informed decision-making in process optimization and chemical planning.

The system 200 includes a data prediction layer 220, which includes a set of databases 222 and a set of predictions 224. In some embodiments, values from the set of databases 222 may be used to provide parameters or inputs to prediction models, ab initio solvers, or other components to generate the set of predictions 224. Some embodiments may use the set of predictions 224 to inform process agent decisions, optimize synthesis protocols, or guide the selection of reaction pathways and experimental conditions for further evaluation. Additionally, the set of predictions 224 may be integrated into workflow optimization routines, scheduling algorithms, or quality assessment modules to improve efficiency, reliability, and product consistency in chemical process development. Furthermore, some embodiments may use the set of predictions 224 to trigger iterative cycles of model refinement, simulation recalibration, or laboratory verification, promoting continuous improvement and adaptive learning.

The system 200 includes a workflow orchestration layer 230, which includes an orchestration system 232 that controls a component group 233 that includes a crystal structure predictor 238, an impurity predictor 234, and a solubility predictor 236. In some embodiments, the output of the crystal structure predictor 238 may then be provided to the impurity predictor 234 and the solubility predictor 236 as an input. Furthermore, operations of the crystal structure predictor 238, the impurity predictor 234, and the solubility predictor 236 may be scheduled to execute on a node N 240. In some embodiments, the orchestration system 232 may control the execution flow and resource allocation for the component group 233, coordinating specialized predictive modeling tasks related to chemical process and material characterization. In some embodiments, the crystal structure predictor 238 may analyze molecular conformations, predict crystallographic arrangements, and generate lattice parameters for synthesized compounds based on computational chemistry and structural modeling algorithms. In some embodiments, the output of the crystal structure predictor 238 may then be provided to the impurity predictor 234 and the solubility predictor 236 as an input, enabling downstream prediction of contaminant formation and physical properties informed by crystalline structure data. In some embodiments, the impurity predictor 234 may evaluate the likelihood of impurity generation or persistence in synthesized products by analyzing crystal structure, reaction conditions, and precursor impurities using data-driven or mechanistic approaches. In some embodiments, the solubility predictor 236 may estimate solubility profiles and solvent compatibility for chemical entities by incorporating crystallographic and physicochemical data, supporting process optimization and formulation development. In some embodiments, the orchestration system 232 may schedule the operations of the crystal structure predictor 238, the impurity predictor 234, and the solubility predictor 236 for execution on a node N 240, maximizing computational efficiency and workflow integration. The output of the component group 233 may be used as one or more predictions for the set of predictions 224.

The system 200 includes an agent layer 250, which includes a set of process agents, including a first process agent 252, a second process agent 254, and a third process agent 256, where each agent may be used to control various operations described in this disclosure. For example, the first process agent 252 may initiate preliminary analysis and route selection for chemical synthesis workflows, aggregating molecular data and recommending feasible starting materials based on internal databases or retrosynthetic algorithms. Furthermore, the second process agent 254 may evaluate and refine the proposed synthetic route by simulating reaction steps, estimating yields, and identifying potential bottlenecks or hazardous conditions using computational modeling tools and predictive analytics. Additionally, the third process agent 256 may finalize the synthesis workflow by incorporating process optimization strategies, scheduling laboratory experimentation or scale-up procedures, and consolidating reporting on yield, purity, and quality metrics for further review or archival in process documentation systems.

The system 200 includes a decision layer 260, which includes a workflow optimizer 262, a quantum optimizer 264, and a schedule optimizer 266. In some embodiments, the workflow optimizer 262 may evaluate and refine process workflows by analyzing the sequence and interdependence of synthetic operations to reduce process complexity, minimize resource usage. In some embodiments, the quantum optimizer 264 may use classical or quantum computing techniques to solve complex optimization problems such as molecular conformer searches, reaction pathway selection, or the determination of energetically favorable states. In some embodiments, the schedule optimizer 266 may generate and adjust execution timetables for laboratory tasks, computational jobs, and resource allocations.

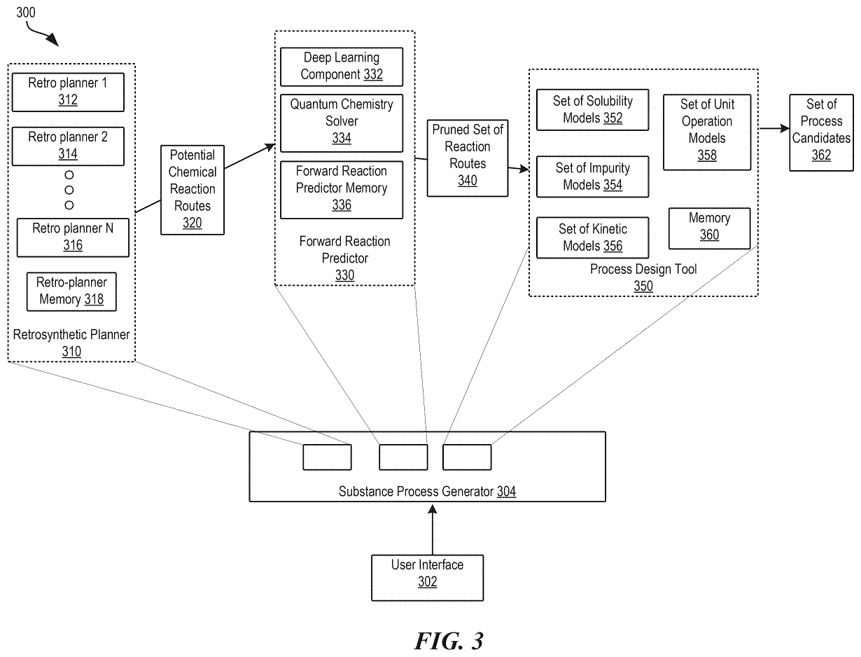

FIG. 3 depicts a conceptual architecture of a system 300 for testing reaction routes using adaptive search retrieval to obtain synthesis process results, in accordance with one or more embodiments. In some embodiments, the system 300 may obtain an initial query via the user interface 302, which may encode target molecules, reaction constraints, or desired outcomes for synthetic planning. The system 300 may send the initial query to a substance process generator 304, which may coordinate subsequent computational modules.

The substance process generator 304 may first direct the initial query to a retrosynthetic planner 310, responsible for decomposing the target molecule(s) into feasible precursor candidates and associated synthetic routes. The retrosynthetic planner 310 may select one or more retro-synthetic solvers based on the chemical structures or metadata present in the initial query or acquired from external data sources, thereby customizing the computation to fit the synthetic challenge. These solvers may include a first retro planner 312, such as the Askcos solver, a second retro planner 314, such as ICSynth, or a third retro planner 316, each offering different algorithms or heuristics tailored to retrosynthetic analysis. In some embodiments, the selected retro-synthetic solvers may obtain additional inputs or parameters from a retro-planner memory 318, allowing the retrieval of historical solution strategies, adaptation to user preferences, and incorporation of context-specific constraints into the retrosynthetic planning process.

In some embodiments, the retrosynthetic planner 310 may aggregate the outputs produced by any utilized retro-planners, such as the first retro planner 312, the second retro planner 314, and the third retro planner 316. The retrosynthetic planner 310 may compile candidate reaction routes generated by each solver into a unified output set. The retrosynthetic planner 310 may group, rank, and filter these potential reaction routes according to criteria such as synthetic feasibility, cost, or alignment with user-specified goals. Furthermore, the aggregated results may be presented to the user via an interactive interface, enabling review and feedback on route quality, novelty, or practicality. In some embodiments, the aggregated results or user feedback for those results may be recorded and analyzed to update the retro-planner memory 318, enhancing future retrosynthetic planning by informing solver selection, prioritization strategies, and adaptive route improvement.

In some embodiments, the retro-synthetic planner may output potential chemical reaction routes 320 to the forward reaction predictor 330, which includes a forward reaction predictor memory 336. The forward reaction predictor 330 may provide the potential chemical reaction routes 320 to at least one of a deep learning component 332 or a quantum chemistry solver 334 (e.g., an ab initio quantum chemistry solver). In some embodiments, the forward reaction predictor 330 may selectively provide the potential chemical reaction routes 320 to a deep learning component 332, which may use neural architectures such as graph neural networks or transformer models to analyze molecular transformations, predict product outcomes, and quantify uncertainty based on large reaction datasets. Alternatively, or additionally, the forward reaction predictor 330 may direct the potential chemical reaction routes 320 to a quantum chemistry solver 334, which may perform ab initio calculations to model electronic structures, reaction energies, and kinetic profiles using advanced quantum algorithms. This orchestration may enable the system to use both data-driven predictions and rigorous physics-based simulations, ensuring comprehensive evaluation of proposed synthetic pathways for further downstream process optimization and experimental planning.

In some embodiments, the forward reaction predictor memory 336 may store solver parameters, learning model parameters, or other values that may be used by the deep learning component 332 or the quantum chemistry solver 334 to process the potential chemical reaction routes 320. The forward reaction predictor memory 336 may include curated reaction templates, tuning coefficients, activation functions, convergence thresholds, and training data for learning models, as well as basis sets, initial configurations, and computational thresholds for quantum solvers. In some embodiments, the forward reaction predictor 330 may utilize these stored parameters and values to efficiently evaluate the predicted outcomes and reliability of candidate reaction routes. Based on model outputs, simulation results, and uncertainty analyses, the forward reaction predictor 330 may generate and output a pruned set of reaction routes 340 that include reactions containing synthetic pathways that satisfy one or more criteria indicating reaction feasibility, yield, selectivity, or safety.

In some embodiments, a process design tool 350 may obtain the pruned set of reaction routes 340. The process design tool 350 may provide each route to a set of solubility models 352, which may predict the solubility of intermediates and final products under varying process conditions to inform solvent selection and separation protocols. Furthermore the process design tool 350 may distribute the pruned set of reaction routes 340 to a set of impurity models 354, which may forecast the generation, persistence, and fate of process-related impurities using mechanistic rules and historical impurity data. The process design tool 350 may also provide the pruned set of reaction routes 340 to a set of kinetic models 356, which may evaluate reaction rates, selectivity, and degradation pathways to support optimization of temperature profiles, residence times, and reactant addition rates. Additionally, the process design tool 350 may provide the pruned set of reaction routes 340 to a set of unit operation models 358 to simulate or model downstream steps such as mixing, filtration, drying, and crystallization, assessing process scalability and robustness. In some embodiments, these different models may access configuration parameters or other relevant values from a memory 360, such as material properties, equipment limitations, historical performance metrics, or validated operational protocols. By integrating outputs from the solubility, impurity, kinetic, and unit operation models, the process design tool 350 may generate a set of process candidates 362, each representing a fully characterized and validated synthesis protocol.

It should be understood that the retro-planner memory 318, the forward reaction predictor memory 336, the memory 360, or some other memory may serve as part of a semantic cache which any workflow node may access, where the semantic cache may store semantic embeddings and associated results for all node executions. For example, a semantic cache may store a record indicating an input embedding (e.g., an input embedding that acts as vector representation of a node's input parameters), node metadata (e.g., node type, tool/solver name, configuration parameters, date, environment state), output representation (processed into a compressed vector or structured schema), execution metrics (e.g., runtime, hardware used, cost, error rates), or prediction accuracy logs.

When retrieving data with a query, some embodiments may use a semantic cache (e.g., a vector database semantic cache) by comparing a vector derived from the query with the vector database of the semantic cache. Some embodiments may apply node-type-specific similarity scoring system (e.g., cosine similarity for embeddings, structural matching for molecule graphs) when performing the search to obtain a similarity score. Some embodiments may then retrieve the most relevant past outputs or metadata (e.g., configuration parameters for a solver) if the similarity score is greater than a similarity score threshold or execute a node with a solver model otherwise.

In some embodiments, the semantic cache may be implemented uniformly across various types of component schemes arranged in the form of a directed acyclic graph (DAG), such as the arrangement shown by the component group 233.

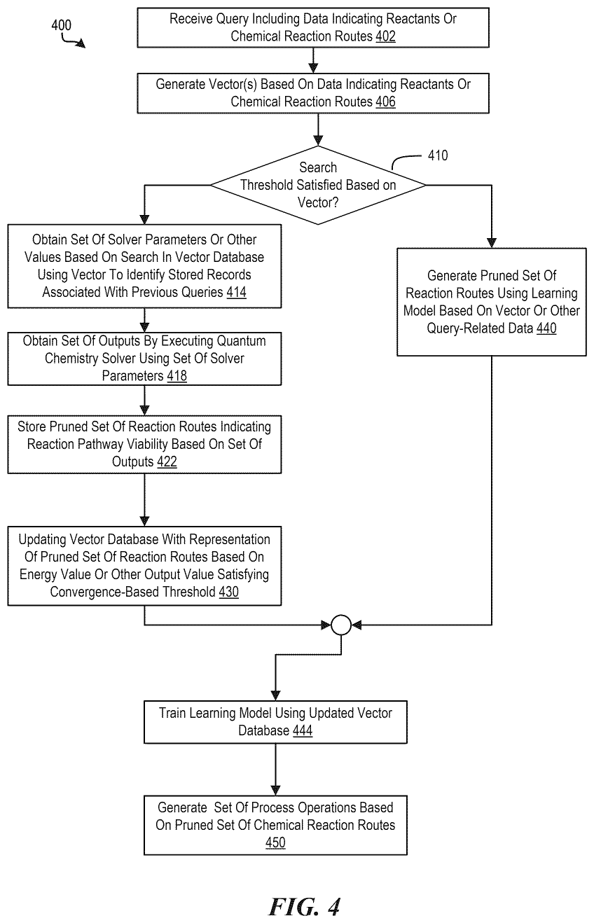

FIG. 4 shows a flowchart of a process 400 for testing reaction routes and generating synthesis processes, in accordance with one or more embodiments. Some embodiments may receive a query including data indicating a reactant or a chemical reaction route, as indicated by block 402. Receiving a query including data that identifies a reactant or specifies a chemical reaction route may include receiving an input designed for chemical process planning. For example, a computer system may prompt a user to enter molecular information or details of a target synthesis pathway into a structured dialog interface. Upon submission, some embodiments may employ agent-based modules and retrosynthetic planning algorithms to analyze the molecule or route and generate an initial set of candidate chemical reactions, presenting these suggestions to the user for review. The computer system may then receive a second query from the user or another agent that contains the set of candidate chemical reactions, allowing some embodiments to perform further operations described in this disclosure.

As described elsewhere in this disclosure, some embodiments may pass a query through different components, where the output of a first component may be used to determine an input query or part of an input query for a second component. The set of retrosynthesis solvers may include at least one of a neural network-based retrosynthesis solver or a Monte Carlo tree-based retrosynthesis solver. For example, a computer system may select a set of retrosynthesis solvers based on a first query or a first vector generated with the first query. The computer system may then analyze a molecule's vector representation and choose both a deep learning retrosynthesis planner and a Monte Carlo tree search module to evaluate possible synthetic pathways. Some embodiments may conduct parallel computations using the set of retrosynthesis solvers to generate multiple chemical reaction routes of the set of chemical reaction routes. For example, the computer system may simultaneously execute both the neural network-based solver and the Monte Carlo tree-based solver, collecting and presenting distinct synthetic routes proposed by each algorithm for further review. By performing such operations, some embodiments may rapidly explore a broader range of synthetic pathways, increasing the likelihood of discovering more efficient, novel, or more feasible chemical reaction routes compared to relying on a single planning algorithm.

Alternatively, or additionally, some embodiments may use additional types of solvers for the set, such as heuristic-guided or rule-based retrosynthesis solvers, to further diversify the generated chemical reaction routes. For example, the computer system may select and operate a heuristic-driven module alongside neural network and Monte Carlo tree algorithms, increasing coverage and improving the prioritization of viable synthesis strategies. Furthermore, some embodiments may aggregate output from the parallel computations using consensus scoring or expert review modules before presenting results for experimental validation. For example, after generating synthetic routes in parallel, the system may score the resulting routes based on predicted feasibility and then forward the top candidates to a verification system for further analysis and approval.

Some embodiments may generate a vector or vectors based on the data indicating the reactant or the chemical reaction route, as indicated by block 406. Some embodiments may generate a vector from a reaction query containing information that specifies a set of chemical reaction routes, incorporating details about reactants and reaction conditions. Reactants may include though not be limited to organic molecules, inorganic compounds, catalysts, or solvents, while reaction conditions may include though not be limited to physical parameters such as temperature, pressure, solvent type, pH level, catalyst selection, reactant concentration, stirring speed, light exposure, humidity, or reaction time. For example, a computer system may receive a reaction query specifying reactants such as toluene, nitric acid, and sulfuric acid, along with a set of chemical reaction routes for nitration of toluene. The reaction conditions may include a temperature of 60° C., pressure of 1.2 atm, use of water as a solvent, pH level of 1, catalyst type as concentrated sulfuric acid, reactant concentration of 0.5 M, stirring speed of 300 rpm, exposure to ambient light, humidity of 50 percent, and reaction time of 45 minutes. The computer system may encode all these details into a vector for further similarity search or predictive analysis.

More generally, even without specifying conditions, some embodiments may generate a vector from a query that includes data indicating a set of reactants and a set of chemical reaction routes. For example, a computer system may receive structured input describing chemical reactants and associated parameters and then apply graph neural networks or embedding algorithms to transform the structured input into an embedding vector of a high-dimensional vector. By generating vectors, some embodiments may encode complex molecular and reaction information into a format suitable for similarity searching, clustering, or predictive modeling. By representing chemical processes and entities as vectors, some embodiments may enable efficient retrieval of closely related reactions, identification of novel pathways, or support real-time decision-making in chemical workflows.

Some embodiments may use transformer-based architectures for generating process embeddings that include reactant details, ordered synthetic steps, or contextual metadata. For example, a computer system may represent reaction workflows by encoding sequences of reactants, reagents, conditions, and previous outcome data, transforming these into vectors through model processing. Some embodiments may analyze the relationships between steps and context using these embeddings, which are stored for efficient searching and recommendation. By applying this approach, the computer system may support improved selection and optimization of synthesis routes by matching workflows to relevant historical examples or published data.

Some embodiments may determine queries and vectors in stages. Some embodiments may determine an initial vector based on user input and then determine a set of chemical reaction routes using this initial vector. Some embodiments may generate a later vector, such as a second-stage vector, by utilizing both the user input and either the original query or the initial vector, incorporating the results derived from the first vector to refine or inform the subsequent vector generation. As an example of the above, some embodiments may provide staged and adaptive chemical route exploration, allowing each step to build on the output and user context from prior steps. Such operations support dynamic interaction and continuous refinement, increasing the accuracy of route prediction and improving the relevance of subsequent computational recommendations.

As an example of the above, a computer system may receive the structural and condition data of a target molecule from a user, embed this data into an initial vector, and use that vector to retrieve or predict possible chemical reaction routes from a database. The computer system may then generate a second vector by combining details of the user input and information from the first-stage routes or outputs, enhancing the query's specificity for a follow-up prediction or retrieval step, such as recommending optimized routes or filtering by additional criteria. The computer system may then provide this second vector to a learning model or use the second vector as to retrieve parameters for a quantum chemistry solver.

Alternatively, or additionally, some embodiments may determine the initial vector from structured molecular descriptors, submitting the initial vector directly to a retrosynthetic prediction module before generating the later vector through aggregation of the module's results and user constraints. For example, a computer system may create a vector from user-specified physicochemical properties, retrieve candidate routes, and then process these candidates with user-supplied priorities to produce a refined follow-up vector for further searches. Furthermore, some embodiments may base the later vector only on user input or external annotations, allowing flexible deviation from the initial query. For example, a computer system may adjust the later vector using feedback or revised requirements, enabling real-time correction or pivoting as new synthetic goals emerge, supporting greater adaptability in complex synthesis planning workflows.

Some embodiments may determine whether a search threshold is satisfied based on a vector, as indicated by block 410. Some embodiments may determine whether a search threshold is satisfied by comparing the similarity between a query vector and candidate vectors in a database using criteria such as Euclidean distance, Manhattan distance, or cosine similarity. The computer system may evaluate these metrics against a predefined cutoff value, treating vectors as sufficiently similar when the computed score is below the set threshold. For example, a computer system may encode query reaction data as a vector [0.82, 1.35, 0.62, 0.97] and compare it to stored vectors, calculating Euclidean distances for each candidate vector. If the minimum distance found is 0.18 and the search threshold is set at 0.25, some embodiments may interpret this as evidence that a sufficiently close past query exists and has been trained on. In response, some embodiments may process information in the query vector with a deep learning system that predicts one or more chemical reaction routes, allowing the computer system to include predicted routes in a pruned set of reaction routes. Otherwise, some embodiments may provide the query or data from the query to a quantum chemistry solver.

As an example, a computer system may perform a vector search by encoding query reaction data as a vector [0.82, 1.35, 0.62, 0.97] and compare it to stored vectors in the database, calculating Euclidean distances for each candidate vector. If the minimum distance found is 0.18 and a search threshold is set at 0.25, some embodiments may return records corresponding to candidates within this distance as part of the search results of the vector search. For example, if a first vector in the database is [0.85, 1.37, 0.65, 0.96], the computed distance may be approximately 0.06, which satisfies the 0.25 threshold and leads to the inclusion of a first record indexed by this first vector in the search results. Conversely, if a second vector in the database is [0.40, 0.99, 1.10, 0.55], the computed distance may be approximately 0.80, which does not satisfy the 0.25 threshold, preventing a second record mapped to the second vector from being returned as part of the search result.

If the search threshold is not satisfied based on the vector, operations of the process 400 may proceed to block 414. Otherwise, operations of the process 400 may proceed to block 440.

Some embodiments may obtain a set of solver parameters or other values based on the search in the vector database using the vector to identify stored records associated with previous queries, as indicated by block 414. Some embodiments may perform a similarity search in a vector database by using a generated vector to identify a set of stored records related to chemical data or process data, e.g., records including information related to chemical reactions, workflows, or process configurations.

Some embodiments may process search results to extract quantum chemistry solver parameters such as basis sets, functional types, convergence tolerances, or molecular geometries aligned with the user input. For example, a computer system may compute a vector embedding from reaction query data involving reactant features such as SMILES strings, temperature, and catalyst composition, then compare this vector against entries in a vector database indexed using Euclidean or cosine similarity. Upon finding closely matching vectors, the computer system may retrieve associated quantum chemistry solver parameters, such as “B3LYP functional,” “6-31G basis set,” and “SCF convergence criteria of 1e-6.” Such solver parameters may enable an accurate or efficient mechanistic simulation of a reaction system. Since vectors may encode high-dimensional reaction, condition, and outcome data, a vector search allows rapid matching and retrieval across thousands of chemical workflows and solver configurations.

As described above, some embodiments may obtain solver parameters stored in records that are retrieved from a search result. In some embodiments, the records and values may be selectively added based on being associated with one or more previous reaction test queries that produced threshold-satisfying energy gaps. For example, a computer system may restrict storage to only those parameters that resulted in successful convergence and sufficiently large energy gaps during prior simulations. This approach reduces the risk of selecting inappropriate solver parameters that either fail to achieve convergence or produce low energy gaps, which may lead to unreliable predictions or poor chemical stability analysis. By focusing on historically validated solver configurations, some embodiments may ensure improved reliability and relevance in quantum chemical simulations for reaction pathway evaluation.

Alternatively, or additionally, some embodiments may perform semantic similarity searches using transformer-generated embeddings that account for text-based descriptions, process notes, or experimental results. For example, a computer system may convert detailed experimental protocols and annotations into process embeddings and use these semantic vectors to search for stored records that include relevant solver parameters, such as input geometry files and force field specifications. This variation improves the ability of some embodiments to locate solver parameters for processes documented in different formats, enhancing the robustness and flexibility of chemical simulation setup and optimization.

As described above, some embodiments may obtain solver parameters stored in records retrieved from a search result, where the records may be records of a set of previous reaction test queries that produced threshold-satisfying energy gaps. As described elsewhere in this disclosure, some embodiments may restrict storage of solver parameter values to only those parameter values that resulted in successful convergence and sufficiently large energy gaps during prior simulations. This approach reduces the risk of selecting inappropriate solver parameters that either fail to achieve convergence or produce low energy gaps, which may lead to unreliable predictions or poor chemical stability analysis. By focusing on historically validated solver configurations, some embodiments may ensure improved reliability and relevance in quantum chemical simulations for reaction pathway evaluation.

Some embodiments may annotate one or more vectors in a vector database with metadata indicating previous outputs or previous solver performance characteristics that are associated with previous executions of a quantum chemistry solver. Indications of previous outputs may include values such as predicted yield, selectivity for a desired product, or calculated energy levels. Indications of previous solver performance characteristics may include metrics such as time to convergence, number of iterations, accuracy of the computed results, or computational resource usage. Some embodiments may obtain quantum chemistry solver parameters by filtering vectors based on the previous outputs or the previous solver performance characteristics. By filtering the vectors using these annotations, some embodiments may retrieve parameter sets that have demonstrated desirable outcomes (such as high yield or selectivity) and solver performance (such as rapid convergence or high accuracy) in prior computational scenarios.

Some embodiments may filter vectors stored in the database using metadata or other data associated with the vectors to isolate a subset of vectors that share one or more filtering categories. Such categories may include various types of values, such as a ligand category, a peptide or peptide sequence, etc. Such operations increase the retrieval efficiency of operations to obtain quantum chemistry solver parameters that match previous outcome compounds (e.g., a ligand category) or specific previous solver performance characteristics such as high yield, convergence speed, accuracy, or target product viability. Previous solver performance characteristics may include quantifiable or descriptive data reflecting how a quantum chemistry solver or similar computational tool performed during previous executions of the solver.

As an example of the operations above, a computer system may update the vector database with annotations such as “yield>85%,” “peptide01,” or “ketone,” directly linking these tags to individual reaction query vectors or parameter sets. When a user seeks solver parameters for a new reaction, the computer system may filter stored vectors to select only those vectors that are linked to the appropriate annotations, increasing the likelihood that retrieved parameters are likely to deliver both technical performance and relevance to user goals when used in a quantum simulator. By performing such operations, some embodiments may provide targeted solver parameter recommendations by reducing trial-and-error and improving simulation outcomes through metadata-driven filtering. Furthermore, such operations may reduce a search space through a vector database by immediately filtering the vector space based on the annotations.

As another example, a computer system may organize the vector database by tagging each stored record with a ligand category such as “peptide ligand,” “metal chelator,” or “aromatic scaffold,” grouping all associated stored vectors under these categories. When a query vector representing a novel metal chelator is received, the computer system may filter the vector database by the ligand category to isolate a subset of vectors labeled “metal chelator.” The computer system may then perform a similarity search within this subset, retrieving the most closely related stored record or records for analysis or recommendation.

Furthermore, outcome metadata linked to one or more vectors may include comprehensive performance summaries, error rates, computational resource use, secondary product formation, or other outcome metadata. For example, a computer system may aggregate execution logs, solution accuracy, and product purities for each solver run, storing these as structured fields within each vector entry for future reference or filtering steps. Furthermore, some embodiments may update annotations dynamically as new solver executions yield updated performance or outcome data. For example, a computer system may append the latest convergence statistics, yield figures, or user feedback to vector metadata as part of an input data stream, supporting continuous refinement of the parameter retrieval process and ongoing adaptation to laboratory or user-specific objectives.

Some embodiments may obtain a set of outputs by executing a quantum chemistry solver using the set of solver parameters, as indicated by block 418. Some embodiments may generate a set of outputs by configuring and executing computational solvers, including but not limited to quantum chemistry solvers, with a set of simulation parameters previously retrieved from search results. In particular, some embodiments may use quantum chemistry solvers such as density functional theory or ab initio methods, applying the retrieved basis sets, functionals, convergence thresholds, or molecular geometries to run electronic structure calculations. For example, a computer system may retrieve parameter values from a vector database, then use these parameters to simulate a reaction system and calculate outputs. The outputs may include values such as a set of reaction rate constants for one or more end products, total energy, electronic states, and molecular orbitals. By using the solver, some embodiments may generate predicted values for information such as chemical properties, reaction feasibility, or mechanistic pathways. Such an approach may improve reliability in chemical modeling through the use of historically validated, threshold-satisfying solver configurations.

Alternatively, or additionally, some embodiments may perform ensemble computations by configuring multiple solvers or simulation variants in parallel using sets of parameters retrieved from different historical cases. For example, a computer system may select parameter sets associated with a range of previously successful reactions and concurrently run geometry optimizations and electronic property calculations using different solvers or settings. Such operations may permit more comprehensive exploration of chemical behaviors and support cross-validation.

As described elsewhere, some embodiments may use a quantum chemistry solver, physics-based solver, or empirical solver when a learning system cannot be sufficiently trusted to produce an accurate outcome. Some embodiments may generate a distance score based on the vector and one or more neighboring vectors stored in the vector database and use the distance score to determine the trustworthiness of a learning model. Some embodiments may then determine that the distance score exceeds a minimum threshold (which may be set to a default value, generated/updated by a user, etc.). For example, a computer system may compute the Euclidean distance between a molecular embedding representing a new compound and several embeddings previously stored in the vector database, quantifying similarities. The computer system may then identify that the distance score exceeds a pre-defined similarity threshold, and, in response to the distance score exceeding the minimum threshold, execute the quantum chemistry solver. By performing such operations, some embodiments may identify possible novel or significantly different chemical entities, thereby directing computing resources towards cases most likely to yield novel results and reducing redundant calculations.

Alternatively, or additionally, some embodiments may generate a distance score using cosine similarity and compare against a dynamically calculated threshold, which adjusts based on the density of data stored in the vector database. For example, a computer system may measure angular similarity between a vector representing a molecular candidate and a cluster of embeddings representing previously solved molecules, adapting the threshold value if the database contains many closely related compounds. Furthermore, some embodiments may execute the quantum chemistry solver using distributed computing resources when the minimum threshold is surpassed, enabling more rapid analysis. For example, once the distance score triggers execution, the computer system may dispatch the solver workload to multiple cloud-based compute nodes or GPUs, accelerating the completion of quantum chemistry calculations for high-priority compounds.

Some embodiments execute a batch of solver executions corresponding with different chemical reaction routes of a plurality of routes. Some embodiments may then execute a quantum chemistry solver by executing a plurality of instances of the quantum chemistry solver. For example, a computer system may generate ten distinct chemical reaction pathways for a target molecule, storing these as the plurality of chemical reaction routes. The computer system may then start several solver instances, each instance independently evaluating electronic structure or chemical properties of one of the proposed routes. Some embodiments may then configure each respective execution of a respective instance of the plurality of instances to correspond with a respective route of the plurality of routes. For example, the computer system may assign each quantum chemistry solver instance a separate input file reflecting a discrete chemical reaction route such that each respective solver runs an analysis of a unique pathway. By performing such operations, some embodiments may improve throughput and reduce decision latency during synthetic pathway evaluation, as multiple chemical reaction routes can be assessed and validated in parallel.

Alternatively, or additionally, some embodiments may employ a queueing mechanism where batch executions of solver instances are scheduled based on available computational resources and route priority. For example, a computer system may defer execution of solvers for lower-priority routes, allowing rapid analysis of those routes flagged by predictive scoring models while remaining within hardware resource limits. Furthermore, some embodiments may aggregate output from each solver instance and automatically rank the chemical reaction routes by computed molecular stability, yield, or feasibility. For example, after the batch execution, the system may compile all results, sort routes according to quantum chemistry calculations, and present top candidates to synthesis planners or expert review modules.

Some embodiments may have a plurality of solver parameter values to choose from for one or more parameters of a solver. Some embodiments may determine a ranked set of solver parameters associated with the set of stored records based on a similarity score between the vector and stored vectors of the vector database. Some embodiments may execute the quantum chemistry solver by providing the ranked set of solver parameters to an interpolation model to determine a target set of solver parameters. One or more types of interpolation models might be used (e.g., regression, weighted average, a more complex optimization algorithm). For example, a computer system may compare the high-dimensional embedding of a current molecule or reaction context to those of previous entries in the vector database, assigning similarity scores using cosine similarity or Euclidean distance and then ranking the historical solver parameter sets accordingly. The computer system may input several top-ranked parameter sets, such as basis set selection, integration grid size, and convergence threshold, into an interpolation model that generates a blended or optimized parameter set tailored for the current quantum chemical simulation. By performing such operations, some embodiments may provide the benefit of reusing and refining historically validated solver settings.

In some embodiments, solver parameters may be of heterogeneous types (e.g., some parameters are numeric, others are categorical). Some embodiments may handle solver parameters heterogeneity by applying a parameter-type-aware interpolation procedure. For example, continuous parameters may be interpolated numerically, and ordinal parameters may be mapped to numeric ranks for interpolation and remapped to ordinal labels. Furthermore, categorical parameters may be selected via weighted frequency voting, and nominal parameters may be mapped to semantic embeddings and interpolated in embedding space. In some embodiments, interpolated candidate values may be validated against hard and soft constraints, and incompatible combinations may be resolved by adjusting to the closest valid configuration within the allowed parameter domain.

Alternatively, or additionally, some embodiments may assign weights to each top-ranked solver parameter set based on the magnitude of the similarity score, allowing the interpolation model to bias toward configurations most closely matched to the current vector. For example, a computer system may compute weighted averages of recommended convergence thresholds (e.g., SCF convergence thresholds) or basis sets, favoring those from the most similar prior contexts, and use the results as the parameters for a solver.

Some embodiments may store a pruned set of reaction routes indicating reaction pathway viability based on the set of outputs, as indicated by block 422. Some embodiments may determine a set of candidate reaction pathways for viability by analyzing outputs from various computational solvers, including quantum chemistry solvers, and then store a pruned selection reflecting the most promising routes. Some embodiments may use a set of reaction rate constants, activation energies, or thermodynamic data obtained directly from the solver outputs or calculated based on these results to assess the likelihood and efficiency of individual reaction pathways. Some embodiments may store, in a reaction route database, a pruned set of chemical reaction routes that indicate pathway viability by evaluating and filtering routes based on calculated reaction rate constants (e.g., a first reaction rate constant for an end product and a second reaction rate constant for a byproduct). By evaluating reaction routes with computational simulations, some embodiments may focus subsequent laboratory resources on only the most productive or feasible synthetic strategies, reducing unnecessary experimentation and optimizing resource allocation.

As an example of the above, a computer system may analyze three candidate reaction routes with predicted reaction rate constants equal to 5.2×10^{-3s}−1, 8.1×10^{-6s}−1, and 4.7×10^{-4s}−1. The computer system may then select only those routes with reaction rate constants greater than 1.0×10^{-4s}−1 for storage in a reaction rate database, where the reaction rate database may also be or include other types of databases or be a part of a larger database. Such operations may increase the likelihood that a retained set of pathways occur in statistically significant amounts as to merit consideration or use.

As described elsewhere in this disclosure, some embodiments may incorporate multiple layers of assessment, combining quantum chemical data with statistical modeling or expert system review to refine the pruning process. Some embodiments may score reaction routes using feasibility indices that integrate calculated energetics, predicted yields, impurity profiles, and historical success rates captured from prior reaction workflow data. For example, a computer system may first use quantum chemistry outputs to calculate a set of rate constants and then apply a Bayesian update based on previously observed empirical yields, filtering out pathways with low viability scores or inconsistent reaction conditions. The remaining routes may be validated through integrated rule-based systems or further simulation, and the computer system may store the most promising options for downstream process planning. Such operations may provide a robust multilayer assessment that reduces risks associated with overreliance on a single evaluation metric and increase the accuracy of viability predictions.

Some embodiments may update the vector database with a representation of the pruned set of reaction routes based on an energy value or other output value satisfying a convergence-based threshold, as indicated by block 430. Some embodiments may update a vector database with representations of a pruned set of reaction routes when an energy value or other output from a computational solver meets a convergence-based threshold. Some embodiments may broaden this update operation to capture routes that satisfy various criteria, including energy values, confidence metrics, predicted yields, or molecular stability indices from quantum or statistical simulations. For example, an energy value may include the HOMO-LUMO gap obtained from a quantum chemistry solver, and an energy gap of 2.9 eV may signal that the route is energetically viable. Some embodiments may selectively store representations in the vector database based on threshold-satisfying energy gaps that help filter out routes with poor stability or incomplete convergence, improving retrieval relevance and downstream predictive analytics. By focusing storage on routes with validated energy or outcome metrics, some embodiments may speed up future similarity searches and support reliable reaction route recommendations.

As an example of the above, a computer system may execute quantum chemistry solvers on a set of candidate reaction routes and obtain energy gap values of 2.1 eV, 0.8 eV, and 3.3 eV for each route. After applying a convergence-based threshold, such as an energy gap greater than 2.0 eV, only the representations of routes with gaps of 2.1 eV and 3.3 eV are converted into high-dimensional vectors and stored within the database. The data associated with pruned routes may include molecular descriptors, reaction conditions, and output scores. This process increases the likelihood that only synthetically viable and electronically stable routes populate the database, while less promising candidates are excluded.

Alternatively, or additionally, some embodiments may combine multiple output metrics, such as reaction yield, energy, impurity formation, and solver uncertainty measurements when determining whether to store results in the vector database. For example, a computer system may assess outcomes from both quantum mechanical and statistical solvers, storing only those routes for which both yield exceeds 80 percent and energy gap surpasses 2.5 eV. In another instance, the computer system may calculate process-embedding scores integrating several attributes and update the database only for top-ranked reaction routes. Practicing this variation leads to a more holistic representation of synthetic route viability in the database, allowing downstream operations to retrieve and analyze multidimensional, high-confidence reaction pathways.

Some embodiments may train a learning model using the updated vector database, as indicated by block 444. Some embodiments may train a learning model using the updated vector database. The trained model may learn complex relationships between chemical entities, reaction conditions, and outcomes. Some embodiments may extend this training to a variety of model architectures, such as graph neural networks, transformer models, or other data-driven approaches, leveraging the full range of data embedded in the vector database. By iteratively training on curated, threshold-satisfying reaction routes and outcomes, some embodiments may improve prediction accuracy, support chemical discovery, and facilitate real-time decision support in synthetic planning.

For example, a computer system may use thousands of high-dimensional vectors representing pruned reaction routes—with molecular descriptors, activation energies, and yield data—to train a graph neural network that predicts feasible synthetic pathways for new compounds. During training, data from the vector database may be used to optimize hyperparameters or validate the learning process. In subsequent workflows, the database may be queried to retrieve solver parameters for newly proposed reactions or to inform predictions by the trained model, providing both historical context and model-driven inference for route selection.

Some embodiments may generate a pruned set of reaction routes using a learning model based on the vector or other query-related data, as indicated by block 440. Some embodiments may generate a pruned set of reaction routes using a learning model based on either a vector representation or other data related to the original query. Some embodiments may extend this operation to use additional query metadata, historical context, and workflow embeddings, optimizing the selection and refinement of chemical pathways for viability and efficiency. For example, a computer system may encode reactant and reaction condition data directly into a high-dimensional vector and use a trained graph neural network to predict and filter synthetic routes, discarding low-probability options. In another example, the computer system may input structured query information, such as experimental settings and historical reaction outcomes associated with the query, to generate a vector and then apply transformer-based models that factor in both current metadata and prior workflow patterns, resulting in a pruned, prioritized set of candidate reaction routes. Furthermore, some embodiments may use both query data and vectors derived from query data as inputs for a learning model used to predict a set of routes.

Some embodiments may use graph neural networks to predict chemical reaction routes, where the molecular structures and reaction networks may be easily represented as graphs. Some embodiments may incorporate transformer architectures to further optimize predictions, as transformers can process sequential and contextual information, allowing detailed encoding of reaction conditions, step order, and experimental context. Some embodiments may incorporate domain-specific adaptations, such as chemical-aware attention mechanisms, physically meaningful feature vectors, or integration of quantum chemistry outputs. Such incorporated elements may improve the ability of a neural network to more accurately learn structure-reactivity relationships. Some embodiments may further customize graph neural network performance by implementing message-passing algorithms tailored for chemical graphs, using richer node and edge features (e.g., atomic descriptors, bond types, reaction role tags), and introducing transfer learning protocols that use pre-trained models on large reaction datasets.

As an example of the above, some embodiments may use a graph neural network model configured with 5 graph convolutional layers, each with 128 hidden units, to predict chemical reaction routes. Furthermore, the computer system may introduce a chemical-aware attention mechanism and augment input features by encoding atomic descriptors such as electronegativity (e.g., 3.44 for oxygen), aromaticity, and reaction role tags for each node and edge. For example, the computer system may optimize model learning by pre-training on a dataset of 200,000 reaction graphs and then fine-tuning with transfer learning using a curated set of 5,000 experimentally verified pharmaceutical synthesis routes. The training process may achieve an accuracy equal to 87 percent and a top three hit rate equal to 94 percent for viable reaction route prediction. These model and optimization steps may improve specificity in predicting complex multi-step reactions and adapt to new classes of chemical transformations.

Some embodiments may use a learning model at an earlier point in the process instead of using a quantum chemistry solver at an earlier point in the process. Some embodiments may train a learning model based on the vector database, wherein the training comprises storing a model context associated with the first vector. Some embodiments may then generate a later-constructed vector based on a later-obtained query. Furthermore, some embodiments may determine that a query vector distance between the first vector and the later-constructed vector satisfies a distance threshold. Additionally, some embodiments may generate a second set of outputs using the learning model by retrieving the model context based on the query vector distance satisfying the distance threshold. By performing such operations, some embodiments may provide the benefit of accelerating process optimization and simulation setup by rapidly reusing or adapting proven configurations from contextually similar cases.

As an example of the operations above, a computer system may use graph neural network architecture to learn vector representations of reaction contexts and save associated solver settings and performance data as the model context, mapped to the first vector in the database. After a user submits a new synthetic target, the computer system may compute an embedding for the new input, generating the later-constructed vector. The system may then calculate a Euclidean or cosine similarity score between the original and new vectors, determining that the value falls below a set limit, thereby indicating substantial similarity in process context. The computer system may then access a historical model context linked to the first vector and automatically retrieve machine learning parameter settings for use by a machine learning model.

Alternatively or additionally, some embodiments may extend the system to refine the retrieved model context by integrating new feedback or outputs from the current query. For example, the computer system may retrieve and reuse solver parameters based on meeting the vector distance threshold, then update the stored context after running the new task, continually enhancing the database and retraining the learning model, so that future queries produce ever more accurate and adaptive recommendations. Furthermore, some embodiments may apply additional domain-specific filters to retrieved model contexts before output generation. For example, after retrieving historical context based on vector similarity, the system may further screen candidate parameter sets to exclude obsolete methods or align with regulatory guidelines before final output generation.

Some embodiments may generate a set of process operations based on the pruned set of chemical reaction routes, as indicated by block 450. Some embodiments may provide the pruned set of chemical reaction routes to additional models, such as a solubility model, a kinetic model, or an impurity prediction model. Based on the results of these models, some embodiments may then select a set of process parameters for the pruned set of reaction routes. In some embodiments, the set of process parameters may indicate environmental parameters such as a temperature, pressure, or pH. Some embodiments may generate a proposed set of process operations based on the set of process parameters and the pruned set of reaction routes. For example, a computer system may analyze kinetic model results for three filtered chemical reaction pathways and select optimal temperature profiles for each pathway to maximize yield while consulting impurity predictions to ensure purity thresholds. The computer system may then construct an operational workflow specifying stepwise temperature, pressure, and pH settings matched with each chemical reaction route, assigning appropriate mixing, heating, and purification instructions for every route. By performing such operations, some embodiments may transform predictive modeling results into precise, actionable process protocols for chemical manufacturing, increasing product quality, safety, and efficiency.

Some embodiments may analyze outputs from solubility models or kinetic models to determine optimal solvent choice and residence time for each pruned reaction route. The computer system may evaluate quantitative solubility predictions, such as maximum solute concentration and temperature-dependent solubility curves, and integrate these findings with kinetic data like rate constants and reaction half-lives to select specific solvents and adjust reaction durations. By tailoring solvent systems and residence times to the unique kinetic and solubility characteristics of each route, some embodiments may increase yield or minimize the formation of unwanted byproducts. For example, when evaluating two competing synthesis routes for a pharmaceutical intermediate, a computer system may select an ethyl acetate-water solvent mixture for a route with favorable solubility and rapid kinetics, assigning a 60-minute residence time at 35° C. In contrast, for a slower pathway, the computer system may recommend dimethyl sulfoxide with a two-hour residence time at 50° C. to ensure complete transformation and maintain solubility for all intermediates.

The above-described embodiments of the present disclosure are presented for purposes of illustration and not of limitation, and the present disclosure is limited only by the claims which follow. Furthermore, it should be noted that the features and limitations described in any embodiment may be applied to one or more other embodiments herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods. Furthermore, not all operations of a flowchart need to be performed.

Furthermore, the computing devices described in this disclosure may be any type of computing device unless otherwise stated, including, but not limited to, a laptop computer, a tablet computer, a hand-held computer, and/or other computing equipment (e.g., a server), including “smart,” wireless, wearable, and/or mobile devices. Furthermore, the embodiments described in this disclosure may include an individual device that performs some or all the operations described in this disclosure. Alternatively, other embodiments may include multiple computing devices acting collectively to perform some or all the operations described in this disclosure.

In some embodiments, the operations described in this disclosure may be implemented in a set of processing devices (e.g., a digital processor, an analog processor, a digital circuit designed to process information, an analog circuit designed to process information, a state machine, and/or other mechanisms for electronically processing information). The processing devices may include one or more devices executing some or all of the operations of the methods in response to instructions stored electronically on one or more non-transitory, machine-readable media (e.g., a set of machine-readable storage media), such as an electronic storage medium. Furthermore, the use of the term “media” may include a single medium or combination of multiple media, such as a first medium and a second medium. One or more non-transitory machine-readable media storing instructions may include instructions included on a single medium or instructions distributed across multiple media. For example, non-transitory media may act as one or more memory, where one or more memory may store program instructions that are written as source files or written in machine-executable program code. The processing devices may include one or more devices configured through hardware, firmware, and/or software to be specifically designed for the execution of one or more of the operations of the methods.

In some embodiments, the various computer systems and subsystems illustrated in FIG. 1 or other figures described in this disclosure may include one or more computing devices that are programmed to perform the functions described herein. The computing devices may include one or more electronic storages (e.g., a set of databases accessible to one or more applications depicted in the system 100), one or more physical processors programmed with one or more computer program instructions, and/or other components. For example, the set of databases may include one or more relational databases. Alternatively, or additionally, the set of databases or other electronic storage used in this disclosure may include one or more non-relational databases.

The computing devices may include communication lines or ports to enable the exchange of information with a set of networks (e.g., a network used by the system 100) or other computing platforms via wired or wireless techniques. The network may include the internet, a mobile phone network, a mobile voice or data network (e.g., a 5G or Long-Term Evolution (LTE) network), a cable network, a public switched telephone network, or other types of communication networks or combination of communication networks. A network described by devices or systems described in this disclosure may include one or more communications paths, such as Ethernet, a satellite path, a fiber-optic path, a cable path, a path that supports internet communications (e.g., IPTV), free-space connections (e.g., for broadcast or other wireless signals), Wi-Fi, Bluetooth, near field communication, or any other suitable wired or wireless communications path or combination of such paths. The computing devices may include additional communication paths linking a plurality of hardware, software, and/or firmware components operating together. For example, the computing devices may be implemented by a cloud of computing platforms operating together as the computing devices.

Each of these devices described in this disclosure may also include electronic storages. The electronic storage may include one or more non-transitory machine-readable media (e.g., storage media) that electronically store information. The storage media of the electronic storages may include one or both of (i) system storage that is provided integrally (e.g., substantially non-removable) with servers or client computing devices, or (ii) removable storage that is removably connectable to the servers or client computing devices via port (e.g., a USB port, a firewire port, etc.) or drive (e.g., a disk drive, etc.). The electronic storages may include one or more optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). An electronic storage may store software algorithms, information determined by the processors, information obtained from servers, information obtained from client computing devices, or other information that enables the functionality as described herein.

The processors may be programmed to provide information processing capabilities in the computing devices. As such, the processors may include one or more of a digital processor, an analog processor, a digital circuit designed to process information, an analog circuit designed to process information, a state machine, and/or other mechanisms for electronically processing information. In some embodiments, the processors may include a plurality of processing units. These processing units may be physically located within the same device, or the processors may represent the processing functionality of a plurality of devices operating in coordination. The processors may be programmed to execute computer program instructions to perform functions described herein of subsystems described in this disclosure or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the different subsystems described herein is for illustrative purposes, and is not intended to be limiting, as any of the subsystems described in this disclosure may provide more or less functionality than is described. For example, one or more of subsystems described in this disclosure may be eliminated, and some or all of its functionality may be provided by other ones of subsystems described in this disclosure. As another example, additional subsystems may be programmed to perform some, or all of the functionality attributed herein to one of the subsystems described in this disclosure.

With respect to the components of computing devices described in this disclosure, each of these devices may receive content and data via input/output (I/O) paths. Each of these devices may also include processors and/or control circuitry to send and receive commands, requests, and other suitable data using the I/O paths. The control circuitry may comprise any suitable processing, storage, and/or I/O circuitry. Further, some or all of the computing devices described in this disclosure may include a user input interface and/or user output interface (e.g., a display) for use in receiving and displaying data. In some embodiments, a display such as a touchscreen may also act as a user input interface. It should be noted that in some embodiments, one or more devices described in this disclosure may have neither user input interface nor displays and may instead receive and display content using another device (e.g., a dedicated display device, such as a computer screen, and/or a dedicated input device, such as a remote control, mouse, voice input, etc.). Additionally, one or more of the devices described in this disclosure may run an application (or another suitable program) that performs one or more operations described in this disclosure.

Although the present invention has been described in detail for the purpose of illustration based on what is currently considered to be the most practical and preferred embodiments, it is to be understood that such detail is solely for that purpose and that the invention is not limited to the disclosed embodiments but, on the contrary, is intended to cover modifications and equivalent arrangements that are within the scope of the appended claims. For example, it is to be understood that the present invention contemplates that, to the extent possible, one or more features of any embodiment may be combined with one or more features of any other embodiment.

As used throughout this application, the word “may” is used in a permissive sense (i.e., meaning having the potential to), rather than a mandatory sense (i.e., meaning must). The words “include,” “including,” “includes,” and the like mean including, but not limited to. As used throughout this application, the singular forms “a,” “an,” and “the” include plural referents unless the context clearly indicates otherwise. Thus, for example, reference to “an element” or “the element” includes a combination of two or more elements, notwithstanding the use of other terms and phrases for one or more elements, such as “one or more.” The term “or” is non-exclusive (i.e., encompassing both “and” and “or”), unless the context clearly indicates otherwise. Terms describing conditional relationships (e.g., “in response to X, Y,” “upon X, Y,” “if X, Y,” “when X, Y,” and the like) encompass causal relationships in which the antecedent is a necessary causal condition, the antecedent is a sufficient causal condition, or the antecedent is a contributory causal condition of the consequent (e.g., “state X occurs upon condition Y obtaining” is generic to “X occurs solely upon Y” and “X occurs upon Y and Z”). Such conditional relationships are not limited to consequences that instantly follow the antecedent obtaining, as some consequences may be delayed, and in conditional statements, antecedents are connected to their consequents (e.g., the antecedent is relevant to the likelihood of the consequent occurring). Statements in which a plurality of attributes or functions are mapped to a plurality of objects (e.g., a set of processors performing steps/operations A, B, C, and D) encompass all such attributes or functions being mapped to all such objects and subsets of the attributes or functions being mapped to subsets of the attributes or functions (e.g., both/all processors each performing steps/operations A-D, and a case in which processor 1 performs step/operation A, processor 2 performs step/operation B and part of step/operation C, and processor 3 performs part of step/operation C and step/operation D), unless otherwise indicated. Further, unless otherwise indicated, statements that one value or action is “based on” another condition or value encompass both instances in which the condition or value is the sole factor and instances in which the condition or value is one factor among a plurality of factors.

Unless the context clearly indicates otherwise, statements that “each” instance of some collection has some property should not be read to exclude cases where some otherwise identical or similar members of a larger collection do not have the property (i.e., each does not necessarily mean each and every). Limitations as to the sequence of recited steps should not be read into the claims unless explicitly specified (e.g., with explicit language like “after performing X, performing Y”) in contrast to statements that might be improperly argued to imply sequence limitations (e.g., “performing X on items, performing Y on the X'ed items”) used for purposes of making claims more readable rather than specifying a sequence. Statements referring to “at least Z of A, B, and C,” and the like (e.g., “at least Z of A, B, or C”), refer to at least Z of the listed categories (A, B, and C) and do not require at least Z units in each category. Unless the context clearly indicates otherwise, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device. Furthermore, unless indicated otherwise, updating an item may include generating the item or modifying an existing item. Thus, updating a record may include generating a record or modifying the value of an already-generated value in a record. Additionally, as used in the specification, “a portion” refers to a part of, or the entirety of (i.e., the entire portion), a given item (e.g., data) unless the context clearly dictates otherwise.

Unless the context clearly indicates otherwise, ordinal numbers used to denote an item do not define the item's position. For example, an item that may be a first item of a set of items even if the item is not the first item to have been added to the set of items or is otherwise indicated to be listed as the first item of an ordering of the set of items. Thus, for example, if a set of items is sorted in a sequence from “item 1,” “item 2,” and “item 3,” the first item of a set of items may be “item 2” unless otherwise stated. Furthermore, a “set” may refer to a singular form or a plural form, such that a “set of items” may refer to one item or a plurality of items.

### Enumerated Embodiments

The present techniques will be better understood with reference to the following enumerated clauses:
1. A method comprising determining a reaction route using a learning model based on a query vector.
2. A method comprising: obtaining a set of parameters by based on a query to identify a set of stored records; executing a prediction model or solver using the set of solver parameters to generate a set of outputs; storing, in a reaction route database, the pruned set of reaction routes indicating reaction pathway viability based on the set of outputs; and determining a set of synthesis process operations based on the pruned set of reaction routes.
3. A method comprising: obtaining a set of solver parameters by based on a query to identify a set of stored records; executing a quantum chemistry solver using the set of solver parameters to test the set of chemical reaction routes and generate a set of outputs comprising a set of reaction rate constants; and storing, in a reaction route database, a pruned set of reaction routes indicating reaction pathway viability based on the set of outputs; and determining a set of synthesis process operations based on the pruned set of reaction routes.
4. A method comprising: generating a vector from a query comprising data indicating a set of reactants and set of chemical reaction routes; obtaining a set of solver parameters by performing a similarity search in a vector database using the vector to identify a set of stored records associated with a set of previous reaction test queries; executing a quantum chemistry solver using the set of solver parameters to test the set of chemical reaction routes and generate a set of outputs comprising a set of reaction rate constants; and storing, in a reaction route database, a pruned set of reaction routes indicating reaction pathway viability based on the set of outputs; and updating the vector database with a representation of the pruned set of reaction routes based on a determination that an output value indicated by the set of outputs satisfies a convergence-based threshold.
5. A method comprising: generating a vector from a reaction query comprising information indicating a set of chemical reaction routes, wherein the information comprises reactants and reaction conditions; obtaining quantum chemistry solver parameters associated with one or more previous reaction test queries associated with threshold-satisfying energy gaps by performing a similarity search in a vector database using the vector to identify a set of stored records comprising: the quantum chemistry solver parameters; executing a quantum chemistry solver using the quantum chemistry solver parameters to test the set of chemical reaction routes and generate a set of outputs comprising reaction rate constants for an end product and a byproduct of the set of chemical reaction routes; storing, in a reaction route database, a pruned set of reaction routes indicating reaction pathway viability by pruning the set of chemical reaction routes based on the reaction rate constants; and updating the vector database with a representation of the pruned set of reaction routes based on a determination that a first energy gap indicated by the set of outputs satisfies a convergence-based threshold indicating an energy gap at convergence.
6. The method of any of the embodiments above, wherein the vector is a later vector, the operations further comprising: determining an initial vector based on a user input; and determining the set of chemical reaction routes based on the initial vector, wherein generating the later vector comprises generating the later vector based on the user input.
7. The method of any of the embodiments above, the operations further comprising annotating one or more vectors of the vector database with one or more indications of previous outputs or previous solver performance characteristics associated with previous executions of the quantum chemistry solver, wherein obtaining the quantum chemistry solver parameters comprises obtaining the quantum chemistry solver parameters by filtering vectors based on the previous outputs or the previous solver performance characteristics.
8. The method of any of the embodiments above, further comprising: generating distance score based on the vector and one or more neighboring vectors stored in the vector database; and determining that the distance score exceeds a minimum threshold, wherein executing the quantum chemistry solver comprises executing the quantum chemistry solver based on the distance score exceeding the minimum threshold.
9. The method of any of the embodiments above, further comprising: selecting a set of retrosynthesis solvers based on the vector, the set of retrosynthesis solvers comprising at least one of a neural network-based retrosynthesis solver or a Monte Carlo tree-based retrosynthesis solver; and conducting parallel computations using the set of retrosynthesis solvers to generate multiple chemical reaction routes of the set of chemical reaction routes.
10. The method of any of the embodiments above, wherein: the set of chemical reaction routes comprises a plurality of routes; executing the quantum chemistry solver comprises executing a plurality of instances of the quantum chemistry solver; and each respective execution of a respective instance of the plurality of instances corresponds with a respective route of the plurality of routes.
11. The method of any of the embodiments above, further comprising: selecting a set of process parameters for the pruned set of reaction routes based on outputs from at least one of a solubility model, a kinetic model, or an impurity prediction model, wherein the set of process parameters indicate at least one of a temperature, pressure, or pH; and generating a proposed set of process operations based on the set of process parameters and the pruned set of reaction routes.
12. The method of any of the embodiments above, wherein the vector is a first vector, further comprising: training a learning model based on the vector database, wherein the training comprises storing a model context associated with the first vector; generating a later-constructed vector based on a later-obtained query; determining that a query vector distance between the first vector and the later-constructed vector satisfies a distance threshold; and generating a second set of outputs using the learning model by retrieving the model context based on the query vector distance satisfying the distance threshold.
13. The method of any of the embodiments above, further comprising: organizing the vector database by grouping stored vectors according to a ligand category associated with each stored record; and performing the similarity search using the vector of the query by first filtering vectors by the ligand category to isolate a subset of vectors; and searching the subset of vectors to access the set of stored records.
14. The method of any of the embodiments above, further comprising determining a ranked set of solver parameters associated with the set of stored records based on a similarity score between the vector and stored vectors of the vector database, wherein executing of the quantum chemistry solver comprises providing the ranked set of solver parameters to an interpolation model to determine a target set of solver parameters.
15. A tangible, non-transitory, machine-readable medium storing instructions that, when executed by a data processing apparatus, cause the data processing apparatus to perform operations comprising those of any of embodiments 1-22.
16. A system comprising one or more processors; and memory storing instructions that, when executed by the processors, cause the processors to effectuate operations comprising those of any of embodiments 1-22.
17. A system comprising means for performing any of embodiments 1-22.

What is claimed is:

1. A system for testing reaction pathway models by obtaining by using a test query vector database to configure ab initio solvers, the system comprising one or more memory storing program instructions that, when executed by one or more processors, performs operations comprising:
generating a vector from a reaction query comprising information indicating a set of chemical reaction routes, wherein the information comprises reactants and reaction conditions;
obtaining quantum chemistry solver parameters associated with one or more previous reaction test queries associated with threshold-satisfying energy gaps by performing a similarity search in a vector database using the vector to identify a set of stored records comprising the quantum chemistry solver parameters;
executing a quantum chemistry solver using the quantum chemistry solver parameters to test the set of chemical reaction routes and generate a set of outputs comprising reaction rate constants for an end product and a byproduct of the set of chemical reaction routes;
storing, in a reaction route database, a pruned set of reaction routes indicating reaction pathway viability by pruning the set of chemical reaction routes based on the reaction rate constants; and
updating the vector database with a representation of the pruned set of reaction routes based on a determination that a first energy gap indicated by the set of outputs satisfies a convergence-based threshold indicating an energy gap at convergence.

2. The system of claim 1, wherein the vector is a later vector, the operations further comprising:
determining an initial vector based on a user input; and
determining the set of chemical reaction routes based on the initial vector, wherein generating the later vector comprises generating the later vector based on the user input.

3. The system of claim 1, the operations further comprising:
annotating one or more vectors of the vector database with one or more indications of previous outputs or previous solver performance characteristics associated with previous executions of the quantum chemistry solver, wherein obtaining the quantum chemistry solver parameters comprises obtaining the quantum chemistry solver parameters by filtering vectors based on the previous outputs or the previous solver performance characteristics.

4. A method for testing reaction pathway models, comprising:
generating a vector from a query comprising data indicating a set of reactants and set of chemical reaction routes;
obtaining a set of solver parameters by performing a similarity search in a vector database using the vector to identify a set of stored records associated with a set of previous reaction test queries;
executing a quantum chemistry solver using the set of solver parameters to test the set of chemical reaction routes and generate a set of outputs comprising a set of reaction rate constants; and
storing, in a reaction route database, a pruned set of reaction routes indicating reaction pathway viability based on the set of outputs; and
updating the vector database with a representation of the pruned set of reaction routes based on a determination that an output value indicated by the set of outputs satisfies a convergence-based threshold.

5. The method of claim 4, further comprising:
generating distance score based on the vector and one or more neighboring vectors stored in the vector database; and
determining that the distance score exceeds a minimum threshold, wherein executing the quantum chemistry solver comprises executing the quantum chemistry solver based on the distance score exceeding the minimum threshold.

6. The method of claim 4, further comprising:
selecting a set of retrosynthesis solvers based on the vector, the set of retrosynthesis solvers comprising at least one of a neural network-based retrosynthesis solver or a Monte Carlo tree-based retrosynthesis solver; and
conducting parallel computations using the set of retrosynthesis solvers to generate multiple chemical reaction routes of the set of chemical reaction routes.

7. The method of claim 4, wherein:
the set of chemical reaction routes comprises a plurality of routes;
executing the quantum chemistry solver comprises executing a plurality of instances of the quantum chemistry solver; and
each respective execution of a respective instance of the plurality of instances corresponds with a respective route of the plurality of routes.

8. The method of claim 4, further comprising:
selecting a set of process parameters for the pruned set of reaction routes based on outputs from at least one of a solubility model, a kinetic model, or an impurity prediction model, wherein the set of process parameters indicate at least one of a temperature, pressure, or pH; and
generating a proposed set of process operations based on the set of process parameters and the pruned set of reaction routes.

9. The method of claim 4, wherein the vector is a first vector, further comprising:
training a learning model based on the vector database, wherein the training comprises storing a model context associated with the first vector;
generating a later-constructed vector based on a later-obtained query;
determining that a query vector distance between the first vector and the later-constructed vector satisfies a distance threshold; and
generating a second set of outputs using the learning model by retrieving the model context based on the query vector distance satisfying the distance threshold.

10. The method of claim 4, further comprising:
organizing the vector database by grouping stored vectors according to a ligand category associated with each stored record; and
performing the similarity search using the vector of the query by first filtering vectors by the ligand category to isolate a subset of vectors; and
searching the subset of vectors to access the set of stored records.

11. The method of claim 4, further comprising determining a ranked set of solver parameters associated with the set of stored records based on a similarity score between the vector and stored vectors of the vector database, wherein executing of the quantum chemistry solver comprises providing the ranked set of solver parameters to an interpolation model to determine a target set of solver parameters.

12. One or more non-transitory, machine-readable media storing program code that, when executed by one or more processors, causes the one or more processors to perform operations comprising:
generating a vector from a query comprising data indicating a set of reactants and set of chemical reaction routes;
obtaining a set of solver parameters by performing a similarity search in a vector database using the vector to identify a set of stored records associated with a set of previous reaction test queries;
executing a quantum chemistry solver using the set of solver parameters to test the set of chemical reaction routes and generate a set of outputs comprising a set of reaction rate constants; and
storing, in a reaction route database, a pruned set of reaction routes indicating reaction pathway viability based on the set of outputs; and
updating the vector database with a representation of the pruned set of reaction routes based on a determination that an output value indicated by the set of outputs satisfies a convergence-based threshold.

13. The one or more non-transitory, machine-readable media of claim 12, wherein the vector is a later vector, the operations further comprising:
determining an initial vector based on a user input; and
determining the set of chemical reaction routes based on the initial vector, wherein generating the later vector comprises generating the later vector based on the user input.

14. The one or more non-transitory, machine-readable media of claim 12, the operations further comprising:
annotating one or more vectors of the vector database with one or more indications of previous outputs or previous solver performance characteristics associated with previous executions of the quantum chemistry solver, wherein obtaining the set of solver parameters comprises obtaining the set of solver parameters based on filtering vectors based on the previous outputs or the previous solver performance characteristics.

15. The one or more non-transitory, machine-readable media of claim 12, further comprising:
generating distance score based on the vector and one or more neighboring vectors stored in the vector database; and
determining that the distance score exceeds a minimum threshold, wherein executing the quantum chemistry solver comprises executing the quantum chemistry solver based on the distance score exceeding the minimum threshold.

16. The one or more non-transitory, machine-readable media of claim 12, further comprising:
selecting a set of retrosynthesis solvers based on the vector, the set of retrosynthesis solvers comprising at least one of a neural network-based retrosynthesis solver or a Monte Carlo tree-based retrosynthesis solver; and
conducting parallel computations using the set of retrosynthesis solvers to generate multiple chemical reaction routes of the set of chemical reaction routes.

17. The one or more non-transitory, machine-readable media of claim 12, wherein:
the set of chemical reaction routes comprises a plurality of routes; and
executing the quantum chemistry solver comprises executing a plurality of instances of the quantum chemistry solver;
each respective execution of a respective instance of the plurality of instances corresponds with a respective route of the plurality of routes.

18. The one or more non-transitory, machine-readable media of claim 12, further comprising:
selecting a set of process parameters for the set of chemical reaction routes based on outputs from at least one of a solubility model, a kinetic model, or an impurity prediction model, wherein the set of process parameters indicate at least one of a temperature, pressure, or pH; and
generating a proposed set of process operations based on the set of process parameters and the set of chemical reaction routes.

19. The one or more non-transitory, machine-readable media of claim 12, wherein the vector is a first vector, further comprising:
training a learning model based on the vector database, wherein the training comprises storing a model context associated with the first vector;
generating a later-constructed vector based on a later-obtained query;
determining that a query vector distance between the first vector and the later-constructed vector satisfies a distance threshold; and
generating a second set of outputs using the learning model by retrieving the model context based on the query vector distance satisfying the distance threshold.

20. The one or more non-transitory, machine-readable media of claim 12, further comprising:
organizing the vector database by grouping stored vectors according to a ligand category associated with each stored record; and
performing the similarity search using the vector of the query by first filtering vectors by the ligand category to isolate a subset of vectors; and
searching the subset of vectors to access the set of stored records.
